(19) United States
(12) Patent Application Publication
(10) Pub. No.: US 2023/0119678 A1
(43) Pub. Date: Apr. 20, 2023

(54) METHOD FOR TESTING XENOTRANSPLANTATION MATERIAL FOR PATHOGEN INFECTION, TEST KIT, AND METHOD FOR PRODUCING XENOTRANSPLANTATION MATERIAL PRODUCT WHICH HAS BEEN EVALUATED FOR PATHOGEN INFECTION

(71) Applicants: Josho Gakuen Educational Foundation, Osaka (JP); PorMedTec Co., Ltd., Kanagawa (JP)

(72) Inventors: Ryo INOUE, Osaka (JP); Akira MOROOKA, Kanagawa (JP)

(73) Assignees: Josho Gakuen Educational Foundation, Osaka (JP); PorMedTec Co., Ltd., Kanagawa (JP)

(21) Appl. No.: 17/502,344

(22) Filed: Oct. 15, 2021

(51) Int. Cl. C12Q 1/689; C12Q 1/686

(52) U.S. Cl. CPC C12Q 1/689; C12Q 1/686

(57) ABSTRACT

To provide a method for testing a xenotransplantation material for pathogen infection, the method allowing redundant procedures in which the same test procedures are repeated using different primers for each pathogen to be at least reduced when a plurality of types of pathogens are tested for; a test kit; and a method for producing a xenotransplantation material product which has been evaluated for pathogen infection. A method for testing a xenotransplantation material for pathogen infection, the method comprising performing genetic tests for infection by pathogens comprising two or more types of bacteria and a virus or a protozoon, in which the genetic test for the two or more types of bacteria comprises using a primer that targets a common base sequence among at least two types of bacteria.

### BACKGROUND OF THE INVENTION

### Field of the Invention

The present invention relates to a method for testing a xenotransplantation material for pathogen infection, the method allowing a plurality of types of pathogens to be conveniently tested for; a test kit; and a method for producing a xenotransplantation material product which has been evaluated for pathogen infection.

### Related Art

Xenotransplantation is carried out from animal species including mammals other than human (e.g., pig, cattle, primate, etc.), but involves a risk of infectious diseases. The most studied animal species as a xenotransplant donor is a pig, but the pig is susceptible to infectious diseases. For example, infectious diseases such as porcine circovirus-associated disease (PCVAD), porcine reproductive and respiratory syndrome (PRRS), porcine epidemic diarrhea (PED), swine fever, and the like have a serious impact on pig populations worldwide.

With respect to the pig which is the most studied animal species as the xenotransplant donor, pigs that have been raised for medical purposes in a closed, well-controlled environment to eliminate the risk of infectious diseases, especially zoonotic diseases are often used as the xenotransplant donor. The pigs are called Designated Pathogen Free (DPF) pigs. The DPF pigs have already been established in New Zealand, the USA, etc. and commercially operated (e.g., Non-Patent Documents 1 and 2). For example, the DPF pigs are to be tested for 27 types of pathogens in New Zealand and, specifically, 10 types of pathogenic bacteria, 15 types of viruses, and 1 type of protozoon are periodically tested for (once or 4 times per year depending on the pathogens to be tested for). In New Zealand, the pathogens are mainly detected by ELISA or a polymerase chain reaction (PCR; including real-time PCR).
Non-Patent Document 1: Wynyard S, Nathu D, Garkavenko 0, et al. Microbiological safety of the first clinical pig islet xenotransplantation trial in New Zealand. Xenotransplantation 2014; 21:309-323.
Non-Patent Document 2: Fishman JA. Infectious disease risks in xenotransplantation. Am J Transplant.2018; 18:1857-1864.

### SUMMARY OF THE INVENTION

When a plurality of types of pathogens (e.g., a plurality of types of bacteria, viruses, protozoa) relating to the above-mentioned animals for xenotransplantation (e.g., pig, cattle, primate, etc.) are tested for, it is not realistic to perform a test by, for example, the PCR for each pathogen from the viewpoint of practicality since additional effort, time, and cost are needed. Therefore, there is a need for a method for testing for a plurality of types of pathogens that cause infection using the smallest number of tests as possible.

For example, when the PCR method is used to test for a plurality of types of pathogens that cause infection using the smallest number of tests as possible, primers to be used in a single PCR reaction need to be designed to have melting temperatures (Tm values) among which differences fall within a predetermined range from the viewpoint of correlation with an annealing step in the PCR reaction. However, as mentioned above, it is more difficult to design the primers to be used in a single PCR reaction so as to have melting temperatures among which differences fall within a predetermined range with the increase in types of the pathogens to be tested for.

The present invention has been made in view of the above-mentioned related art and an object of the present invention is to provide a method for testing a xenotransplantation material for pathogen infection, the method allowing a plurality of types of pathogens to be conveniently tested for; a test kit; and a method for producing a xenotransplantation material product which has been evaluated for pathogen infection.

The present inventors have focused on, in a test of a xenotransplantation material for pathogen infection, which type of virus or protozoon is detected is important for viruses or protozoa, while, for bacteria, a xenotransplantation material which has been infected with any bacterium should be avoided as a transplantation material regardless of type. Therefore, the present inventors have found that the test can be simplified by focusing on detection of the presence or absence of bacteria rather than identification of types of bacteria using at least one primer targeting a common nucleotide sequence among at least two types of bacteria. The present invention has been completed based on the above finding. That is, the present invention is as follows.

<1> A method for testing a xenotransplantation material for pathogen infection, the method including performing genetic tests for infection by pathogens including two or more types of bacteria and a virus or a protozoon, wherein the genetic test for the two or more types of bacteria comprises using at least one primer that targets a common base sequence among at least two types of bacteria.

<2> The method according to <1>, in which the common base sequence is a sequence included in a conserved region of bacterial 16S rRNA.

<3> The method according to <2>, in which the conserved region is a conserved region at least common to 16S rRNAs of Leptospira species, Mycoplasma species, Campylobacter species, Yersinia species, Escherichia species, and Salmonella species.

<4> The method according to <2>, in which the conserved region is a region including V3 and V4 regions.

<5> The method according to any one of <1> to <4>, in which one primer pair is used in the genetic test for the two or more types of bacteria.

<6> The method according to any one of <1> to <5>, in which the genetic test for infection by the two or more types of bacteria and the genetic test for infection by the virus or the protozoon are performed by a PCR method with the same thermal cycle conditions using primers among which differences in melting temperature are 15° C. or less in any combination.

<7> The method according to any one of <1> to <6>, in which the pathogen infection causes a zoonotic disease.

<8> The method according to any one of <1> to <:7>, in which performing the genetic test for infection by the virus is a testing for infection by five or more types of viruses by a PCR method using at least one primer pair that selectively binds to respective viral nucleic acids, or performing the genetic test for infection by the protozoon is a testing for infection by two or more types of protozoa by a PCR method using at least one primer pair that selectively binds to respective protozoan nucleic acids.

<9> The method according to any one of <1> to <8>, in which the xenotransplantation material is a pig-derived xenotransplantation material.

<10> The method according to any one of <1> to <9>, in which the virus includes porcine reproductive and respiratory syndrome virus, porcine circovirus type 2, swine influenza virus type H1N1, porcine lymphotropic herpesvirus, porcine cytomegalovirus, and hepatitis E virus, or the protozoon includes Toxoplasma species.

<11> A test kit, including: at least one primer that targets a common base sequence among at least two types of bacteria; and at least one primer that selectively binds to a nucleic acid of at least one type of a virus or a protozoon.

<12> A method for producing a xenotransplantation material product which has been evaluated for pathogen infection, the method including: performing the method according to any one of <1> to <10>.

The present invention can provide a method for testing a xenotransplantation material for pathogen infection, the method allowing a plurality of types of pathogens to be conveniently tested for; a test kit; and a method for producing a xenotransplantation material product which has been evaluated for pathogen infection.

### BRIEF DESCRIPTION OF THE DRAWINGS

FIG. 1A shows electrophoresis results of PCR products of 15 types of viruses detected by Standard PCR;

FIG. 1B shows electrophoresis results of PCR products of 15 types of viruses detected by Nested PCR;

FIG. 2A shows detection results of pathogens for evaluating a PCR testing system using clinical specimens, via melting curves as the detection results of porcine lymphotropic herpesvirus; FIG. 2B shows detection results of pathogens for evaluating a PCR testing system using clinical specimens, via an electropherogram as the detection results of bacteria;

FIG. 2C shows detection results of pathogens for evaluating a PCR testing system using clinical specimens, via melting curves as the detection results of porcine cytomegalovirus;

FIG. 2D shows detection results of pathogens for evaluating a PCR testing system using clinical specimens, via an electropherogram as the detection result of getah virus; and FIG. 3 shows electrophoresis results of PCR products of cytomegalovirus using a particular primer.

### DETAILED DESCRIPTION OF THE INVENTION

Although embodiments of the present invention will be hereinafter described in detail, the present invention is not limited to embodiments below in any way and can be implemented with modifications as appropriate within the scope of the object of the present invention.

<<Method for Testing Xenotransplantation Material for Pathogen Infection>>

A first aspect of the present invention is a method for testing a xenotransplantation material for pathogen infection, the method including a step of performing a genetic test for infection by pathogens including two or more types of bacteria and a virus or a protozoon, in which the genetic test for the two or more types of bacteria comprises using at least one primer that targets a common base sequence among at least two types of bacteria. The first aspect can at least reduce redundant procedures in which the same test procedures are repeated using different primer sets (hereinafter may be referred to as “primer pair”: a pair of Forward primer (F primer) and Reverse primer (R primer)) for each pathogen by using at least one primer that targets a common base sequence among at least two types of bacteria. The above-mentioned step of performing the genetic test for the two or more types of bacteria and the above-mentioned step of performing the genetic test for the virus or the protozoon are both preferably steps of testing by a PCR method.

In the present invention, the PCR method may or may not be a standard PCR (Standard PCR) and may or may not be a so-called RT-PCR method in which a complementary cDNA is synthesized by a reverse transcriptase from RNA serving as a template and then subjected to a PCR procedure from the viewpoint of detection of RNA viruses (such as influenza virus). Furthermore, the PCR method may or may not be a real-time PCR method (Real-time PCR) from the viewpoint of increasing quantitativity. Moreover, the PCR method may or may not be a modified method such as a nested PCR method (Nested PCR) from the viewpoint of improving specificity and yield.

Examples of the xenotransplantation material to be tested include materials derived from any animals for xenotransplantation and examples of the animals for xenotransplantation include pigs, cattle, sheep, primates (e.g., chimpanzee and monkey), and the like, with the pigs being preferred. Examples of the xenotransplantation material include organs (such as liver, kidney, pancreas, and heart), blood, urine, sweat, and the like, with the organs or the blood being preferred. The animals for xenotransplantation are not particularly limited, and may be wild-type or animals that have been raised for medical purposes in a closed, well-controlled environment to eliminate the risk of infectious diseases, especially zoonotic diseases (e.g., DPF pigs in the case of the pigs). Examples of the DPF pigs include pigs that have been raised with an UiR method.

In the first aspect, the number of types of the bacteria to be genetically tested for is not particularly limited as long as it is two or more, but is preferably 5 or more, more preferably 10 or more, and further preferably 15 or more. The upper limit of the number of types of the bacteria to be genetically tested for is not particularly limited, but is, for example, 100 or less or 80 or less. Among all types of the bacteria to be genetically tested for, the types of the bacteria to be genetically tested for using the primer targeting a common base sequence among at least two types of bacteria preferably account for 50% or more, more preferably 70% or more, and further preferably 90% or more. The primer to be used for performing the genetic test for the two or more types of bacteria are not particularly limited as long as the primer includes a common base sequence among at least two types of bacteria, and may be, for example, those including any other sequences in addition to the above-mentioned sequence. Base lengths of the primer are not particularly limited as long as the primer serves as a primer capable of selectively binding to the common base sequence among at least two types of bacteria, but are, for example, 10 to 50 bases in length, preferably 12 to 40 bases in length, and more preferably 15 to 30 bases in length. The number of pairs of the primer to be used for performing the genetic test for the two or more types of bacteria may be 2 or more (e.g., 2, 3, 4, . . . , 10), but one pair of primers is preferably used from the viewpoint of avoiding complexity of the test. As the primer to be used for performing the genetic test for the two or more types of bacteria, the primer that targets a common base sequence among at least two types of bacteria may or may not be used in combination with a primer that selectively binds to nucleic acids of other bacteria (e.g., bacteria not having the common base sequence) in order to detect the other bacteria.

A Tm value of the primer to be used for performing the genetic test for the two or more types of bacteria is preferably 50° C. or more and more preferably 55° C. or more from the viewpoint of avoiding non-specific annealing to genomic genes (e.g., pig genomic genes). Furthermore, the upper limit of the Tm value of the primer is not particularly limited, but is, for example, 80° C. or less and preferably 75° C. or less. The primer targeting a common base sequence among at least two types of bacteria is preferably a primer targeting a common base sequence among three or more types of bacteria, more preferably a primer targeting a common base sequence among five or more types of bacteria, further preferably primers targeting a common base sequence among 10 or more types of bacteria, particularly preferably a primer targeting a common base sequence among 15 or more types of bacteria, and most preferably a primer targeting a common base sequence among 20 or more types of bacteria. The upper limit of the number of types of the bacteria is not particularly limited, but is, for example, 100 or less or 80 or less.

A 16S ribosomal RNA (rRNA) may be common to all bacteria. The 16S rRNA has a highly conserved region across bacteria (e.g., conserved regions C1 to C9). The common base sequence is preferably a sequence included in a conserved region of bacterial 16S rRNA from the viewpoint of more securely achieving the effects of the present invention. The conserved region is preferably a region including V3 and V4 variable regions (or a plurality of regions between which the V3 and V4 variable regions are sandwiched) from the viewpoint of high conservation among a plurality of types of bacteria. A primer pair targeting the region including V3 and V4 variable regions is exemplified by a primer pair described below, but a primer pair according to the present invention is not limited thereto.

| (SEQ ID NO: 1) | |
|---|---|
| 341F: | 5′-CCTACGGGNGGCWGCAG-3′ |
| (SEQ ID NO: 2) | |
| 805R: | 5′-GACTACHVGGGTATCTAATCC-3′ |

The pathogens to be tested for including two or more types of bacteria and a virus or a protozoon are preferably pathogens including two or more types of bacteria and both of a virus and a protozoon. Furthermore, the pathogens to be tested for may or may not include fungi, for example, dermatophyte such as Trichophyton species. Examples of the bacteria, viruses, and protozoa to be tested for include bacteria, viruses, and protozoa causing zoonotic diseases, animal infectious diseases (e.g., porcine infectious diseases), or human infectious diseases, with the bacteria, viruses, and protozoa causing zoonotic diseases being preferred. Examples of the two or more types of bacteria to be tested for include at least two types of bacteria selected from specific examples described below. Specific examples: Yersinia species, Bordetella bronchiseptica, Clostridium species, Mycobacterium tuberculosis, Mycobacterium bovis, Mycobacterium avium, Salmonella species, Escherichia coli, Bacillus anthracis, Erysipelothrix rhusiopathiae, Pasteurella species, Brachyspira hyodysenteriae, Haemophilus species, Staphylococcus species, Brucella species, Haemobartonella species, Mycoplasma species, Listeria species, Actinobacillus species, Streptococcus species, Pseudomonas aeruginosa, Actinomyces suis, Campylobacter species, Actinobacillus species, Chlamydia species, Coxiella species, Lawsonia species, Leptospira (Leptopira) species, Eperythrozoon suis.

The at least two types of bacteria are preferably at least two types selected from the group consisting of Leptospira species, Mycoplasma species, Campylobacter species, Yersinia species, Escherichia species, and Salmonella species and the conserved region is preferably a conserved region at least common to 16S rRNAs of Leptospira species, Mycoplasma species, Campylobacter species, Yersinia species, Escherichia species, and Salmonella species.

Specific examples of the viruses to be tested for include porcine circovirus type 1, porcine circovirus type 2, porcine lymphotropic herpesvirus type 1, porcine lymphotropic herpesvirus type 2, porcine cytomegalovirus, encephalomyocarditis virus, porcine parvovirus, porcine rotavirus A, porcine rotavirus B US variant, porcine rotavirus B Vietnam variant, porcine rotavirus C, mammalian reovirus, porcine enterovirus, porcine hemagglutinating encephalomyelitis virus, porcine reproductive and respiratory syndrome virus, hepatitis E virus, porcine transmissible gastroenteritis virus, swine influenza virus type H1N1, swine influenza virus type H3N2, porcine respiratory coronavirus, swine fever virus, swine poxvirus, African swine fever virus, Aujeszky's disease virus, getah virus, Japanese encephalitis virus, porcine epidemic diarrhea virus, swine vesicular disease virus, vesicular exanthema of swine virus, vesicular stomatitis virus New Jersey variant, vesicular stomatitis virus Indiana variant, foot-and-mouth disease virus, rabies virus 1, rabies virus 2, rabies virus 3, rabies virus 4, rabies virus 5, rabies virus 6, rabies virus 7, porcine adenovirus, porcine astrovirus 1, porcine astrovirus 2, porcine astrovirus 3, porcine astrovirus 4, porcine astrovirus 5, Menangle virus, Nipah virus, hantavirus, Eastern equine encephalitis virus 1, Eastern equine encephalitis virus 2, Eastern equine encephalitis virus 3, Eastern equine encephalitis virus 4, Western equine encephalitis virus, Venezuelan equine encephalitis virus, bornavirus, apoi virus, polyomavirus, bovine viral diarrhea virus type 1, bovine viral diarrhea virus type 2, bovine viral diarrhea virus type 3, infectious bovine rhinotracheitis virus, porcine Torque teno virus type 1, porcine Torque teno virus type 2, sapovirus type 3, sapovirus type 6, sapovirus type 7a, sapovirus type 7b, sapovirus type 7c, sapovirus type 8, sapovirus type 9, sapovirus type 10, norovirus type A, norovirus type B, porcine rubulavirus, calicivirus, spumavirus, porcine gammaherpesvirus, porcine endogenous retrovirus, but the porcine endogenous retrovirus may not be included from the viewpoint of low pathogenicity. The viruses to be tested for preferably at least includes the porcine reproductive and respiratory syndrome virus, the porcine circovirus type 2, the swine influenza virus type H1N1, the porcine lymphotropic herpesvirus, the porcine cytomegalovirus, and the hepatitis E virus.

Specific examples of the protozoa to be tested for include Toxoplasma species, Coccidia species, Balantidium species, Cryptosporidium species, Sarcocystis species, Babesia species, Trypanosoma species, Ascaris suum, Toxocara species, Echinococcus species, Hyostrongylus rubidus, Gigantorhynchus gigas, Metastrongylus species, Strongyloides species, Taenia solium, Ciliophora species, Trichostrongylus species, Trichuris suis, other ectoparasites, and the protozoa to be tested for preferably at least include the Toxoplasma species.

The step of performing the genetic test for the virus or the protozoon is preferably a step of testing by the PCR method using primer pairs selectively (preferably specifically) binding to respective nucleic acids (such as DNA and RNA) of at least one type of a virus or a protozoon. The primer capable of selectively binding to respective nucleic acids of at least one type of a virus or a protozoon is not particularly limited and may be that including any other sequences in addition to sequences which selectively bind as mentioned above. Base lengths of the primer are not particularly limited as long as the primer serves as the primer capable of selectively binding to respective nucleic acids of at least one type of a virus or a protozoon, but are, for example, 10 to 50 bases in length, preferably 12 to 40 bases in length, and more preferably 15 to 30 bases in length. The Tm value of the primer is preferably 50° C. or more and more preferably 55° C. or more from the viewpoint of avoiding non-specific annealing to genomic genes (e.g., pig genomic genes). Furthermore, the upper limit of the Tm value of the primer is not particularly limited, but is, for example, 80° C. or less and preferably 75° C. or less. Furthermore, differences in melting temperature among all primers to be used for testing for infection by the bacteria and testing for infection by the virus or the protozoon are preferably 25° C. or less (more preferably 20° C. or less, further preferably 15° C. or less, and particularly preferably 12° C. or less). The differences in melting temperature among all primers are preferably as small as possible, and the lower limit of the differences in melting temperature is not particularly limited, but is, for example, 10° C. or more, 8° C. or more, 6° C. or more, 5° C. or more, 3° C. or more, 1° C. or more, etc., and ideally 0° C.

The step of performing the genetic test for infection by at least one or more type of viruses is preferably a step of testing for infection by five or more types (preferably 10 or more types, more preferably 20 or more types, further preferably 25 or more types) of viruses by the PCR method using primer pairs that selectively bind to respective viral nucleic acids. The upper limit of the number of types of the viruses is not particularly limited, but is, for example, 100 or less or 50 or less. Furthermore, the step of performing the genetic test for infection by at least one or more type of protozoa is preferably a step of testing for infection by two or more types (preferably three or more types) of protozoa by the PCR method using primer pairs that selectively bind to respective protozoan nucleic acids. The upper limit of the number of types of the protozoa is not particularly limited, but is, for example, 10 or less or 5 or less.

The primer pairs that are capable of selectively binding to viral nucleic acids are exemplified by primer pairs described below, but primer pairs according to the present invention are not limited thereto.

### (Primer Pair Targeting Porcine Reproductive and Respiratory Syndrome Virus)

| (SEQ ID NO: 3) | |
|---|---|
| F: | 5′-CACTGTGGAGTTTAGTTTGC-3′ |
| (SEQ ID NO: 4) | |
| R: | 5′-CACACGGTCGCCCTAATTGAATA-3′ |

### (Primer Pair Targeting Porcine Circovirus (Type 2))

| (SEQ ID NO: 5) | |
|---|---|
| F: | 5′-CCAGGAGGGCGTTGTGACT-3′ |
| (SEQ ID NO: 6) | |
| R: | 5′-CGCTACCGTTGGAGAAGGAA-3′ |

### (Primer Pair Targeting Swine Influenza Virus (Type H1N1))

| (SEQ ID NO: 7) | |
|---|---|
| F: | 5′-AGGATTTGTGTTCACGCTCA-3′ |
| (SEQ ID NO: 8) | |
| R: | 5′-GCATTTTGGACAAAGCGTCT-3′ |

### (Primer Pair Targeting Porcine Lymphotropic Herpesvirus (Types 1 and 2))

| (SEQ ID NO: 9) | |
|---|---|
| F: | 5′-AGGACCTGCATAAGTATCCTCAA-3′ |
| (SEQ ID NO: 10) | |
| R: | 5′-CAGGGCCAGAACTAATCAAAA-3′ |

### (Primer Pair Targeting Porcine Cytomegalovirus)

| (SEQ ID NO: 11) | |
|---|---|
| F: | 5′-AATGCGTTTTACGGCTTCAC-3′ |
| (SEQ ID NO: 12) | |
| R: | 5′-ACGCCGCTAGAGGGAGAC-3′ |

### (Primer Pair Targeting Hepatitis E Virus (ORF1) (See Non-Patent Document 1))

| (SEQ ID NO: 13) | |
|---|---|
| F: | 5′-CTGGCATYACTACTGCYATTGAGC-3′ |
| (SEQ ID NO: 14) | |
| R: | 5′-CCATCRARRCAGTAAGTGCGGTC-3′ |

The primer pair that is capable of selectively binding to protozoan nucleic acid is exemplified by a primer pair described below, but a primer pair according to the present invention is not limited thereto.

(Primer Pair Targeting Toxoplasma gondii)

| (SEQ ID NO: 15) | |
|---|---|
| F: | 5′-CTAGGGCACCCTTACTGCAA-3′ |
| (SEQ ID NO: 16) | |
| R: | 5′-CTGCTGGATCTCTTCCCTTG-3′ |

In the step of performing the genetic test for the two or more types of bacteria and the step of performing the genetic test for the virus or the protozoon, detection of infection by the two or more types of bacteria and detection of infection by the virus or the protozoon are not particularly limited as long as nucleic acids of the two or more types of bacteria and a nucleic acid of the virus or the protozoon are detected with statistical significance. For example, the above-mentioned nucleic acids are detected in an amount of 1.1 times or more, preferably 1.2 times or more, and more preferably 1.5 times or more as much as a control. The upper limit of the detection of the nucleic acids is not particularly limited, but is, for example, 10 times or less. The control may be a measurement value obtained from an animal (e.g., pig) prior to viral infection or a statistical value or range obtained by previously collecting from specimens of a healthy animal (e.g., healthy pig) group.

The above-mentioned infection may or may not be detected based on a label intensity (e.g., absorbance, enzyme label intensity, fluorescence intensity, ultraviolet intensity, radiation intensity, etc.) measured on a label substance (e.g., label antibody).

In the step of performing the genetic test for the two or more types of bacteria and the step of performing the genetic test for the virus or the protozoon, thermal cycle conditions in the PCR (e.g., temperature, time, number of cycles, etc.) are not particularly limited, but the PCR can be performed by appropriately setting a temperature and time at which a denaturation reaction in which double-strand nucleic acids are separated into single strands, an annealing reaction, and a nucleic acid elongation reaction by a nucleic acid synthase such as a DNA polymerase take place and repeating these reactions for tens of cycles, taking common general technical knowledge in the art into consideration. Conditions for the denaturation reaction are not particularly limited, but, for example, a target nucleic acid may be heated at 90° C. to 100° C. for several tens of seconds to several minutes. The annealing reaction in which a primer and a target nucleic acid are placed under conditions under which the primer and the target nucleic acid are hybridized with each other is also not particularly limited, but, for example, a product of the denaturation reaction may be, for example, rapidly cooled and left to stand at 50° C. to 70° C. (preferably 52° C. to 65° C.) for several tens of seconds to several minutes.

The nucleic acid elongation reaction is also not particularly limited, but, for example, may be performed at a temperature suitable for enzyme activity of a nucleic acid synthase such as a DNA polymerase using the nucleic acid synthase.

The step of performing the genetic test for the two or more types of bacteria and the step of performing the genetic test for the virus or the protozoon are preferably simultaneously performed from the viewpoint of testing in the smallest number of times as possible. From the viewpoint of testing in the smallest number of times as possible, the two or more types of bacteria and the virus or the protozoon are preferably genetically tested for by the PCT method with the same thermal cycle conditions. Furthermore, differences in melting temperature among all primers to be used for testing for infection by the bacteria and testing for infection by the virus or the protozoon is preferably 25° C. or less (more preferably 20° C. or less, further preferably 15° C. or less, and particularly preferably 12° C. or less). The differences in melting temperature among all primers are preferably as small as possible, and the lower limit of the differences in melting temperature is not particularly limited, but are, for example, 10° C. or more, 8° C. or more, 6° C. or more, 5° C. or more, 3° C. or more, 1° C. or more, etc., and ideally 0° C. A time point and frequency at which the test method according to the first aspect is performed are not particularly limited.

### <<Test Kit>>

A second aspect of the present invention is a test kit, including a primer that targets a common base sequence among at least two types of bacteria; and a primer that selectively binds to a nucleic acid of at least one type of a virus or a protozoon. Specific and preferred examples of the primer targeting a common base sequence among at least two types of bacteria are the same as those mentioned for the first aspect. Specific and preferred examples of the primer that selectively binds to a nucleic acid of at least one type of a virus or a protozoon are the same as those mentioned for the first aspect. The test kit according to the second aspect may or may not include any component for performing the PCR such as a DNA polymerase, dNTP, a PCR reaction buffer, etc. Furthermore, the test kit according to the second aspect may include a container which may contain the above-mentioned components. The test kit according to the second aspect may or may not be a kit in which the primer that targets a common base sequence among at least two types of bacteria and the primer that selectively binds to a nucleic acid of at least one type of a virus or a protozoon are immobilized on any substrate (e.g., plastic substrate) (preferably in an array format).

<<Method for Producing Xenotransplantation Material Product which has been evaluated for pathogen infection>>

A third aspect of the present invention is a method for producing a xenotransplantation material product which has been evaluated for pathogen infection, the method including performing the test method according to the first aspect. A material which is detected to be infected with a pathogen including a bacterium, virus, or protozoon by the above-mentioned step is evaluated as unavailable for a xenotransplantation material product, and a material which is not detected to be infected with a pathogen including a bacterium, virus, or protozoon by the above-mentioned step is evaluated as available for a xenotransplantation material product. Examples of the xenotransplantation material product include organ (such as liver, kidney, pancreas, and heart) products, blood products, urine products, sweat products, etc., with the organ products or the blood products being preferred. The xenotransplantation material product obtained by the second aspect has less risk of pathogen infection and may be suitably used for xenotransplantation.

### Examples

Hereinafter, the present invention will be more specifically described with reference to Examples of the present invention, but the present invention is not limited thereto and variously applied without departing from the technical idea of the present invention.

### <1. Establishment of System for Detecting Pathogen by PCR>

### (1.1. Primer)

TABLE 1-1
Table. Specific primers targeting pathogens

| | | | Size of | Melting | |
|---|---|---|---|---|---|
| Reaction composition | | | PCR | temperature | |
| and condition used for | | | product | of PCR | Positive |
| PCR | Virus name | Type of primer used | (bp) | product (° C.) | control |
| | RNA virus | | | | |
| real-time PCR(1) | Apoi virus | F | 320 | 87 | D |
| | | R | | | |
| nested PCR(1) | Hepatitis E virus(ORF1) | F (SEQ ID NO 13) | 418 | | D |
| | | R (SEQ ID NO 14) | | | |
| | | nested F | 287 | | |
| | | nested R | | | |
| nested PCR(1) | Hepatitis E virus(ORF2) | F | 197 | | D |
| | | R | | | |
| | | nested F | 145 | | |
| | | nested R | | | |
| standard PCR(2) | Bovine viral diarrhea virus(type 1) | F | 64 | 83 | D |
| | | R | | | |
| standard PCR(2) | Bovine viral diarrhea virus(type 2) | F | 73 | 83 | D |
| | | R | | | |
| standard PCR(2) | Bovine viral diarrhea virus(type 3) | F | 59 | 79 | D |
| | | R | | | |
| real-time PCR(2) | Rabies virus(type 4) | F | 62 | 78 | D |
| | | R | | | |
| real-time PCR(3) | Rabies virus(type 5) | F | 110 | 79 | D |
| | | R | | | |
| real-time PCR(2) | Rabies virus(type 6) | F | 63 | 81 | D |
| | | R | | | |
| nested PCR(2) | Rabies virus(type 7) | F | 373 | | D |
| | | R | | | |
| | | nested F | 259 | | |
| | | nested R | | | |
| standard PCR(1) | Getah virus | F | 380 | | C |
| | | R | | | |
| real-time PCR(2) | Foot-and-mouth disease virus | F | 88 | 83 | D |
| | | R | | | |
| real-time PCR(2) | Respiratory coronavirus | F | 113 | 78 | D |
| | | R | | | |
| real-time PCR(2) | Vesicular stomatitis virus(New jersey variant) | F | 73 | 80 | D |
| | | R | | | |
| real-time PCR(2) | Western equine encephalitis virus | F | 67 | 83 | D |
| | | R | | | |
| nested PCR(1) | Hemagglutinating encephalomyelitis virus | F | 682 | | D |
| | | R | | | |
| | | nested F | 472 | | |
| | | nested R | | | |
| real-time PCR(2) | Swine fever virus | F | 62 | 83 | D |
| | | R | | | |
| real-time PCR(2) | Nipah virus | F | 99 | 84 | D |
| | | R | | | |
| standard PCR(3) | Japanese encephalitis virus | F | 142 | | C |
| | | R | | | |
| real-time PCR(2) | Encephalomyocarditis virus | F | 104 | 79 | D |
| | | R | | | |
| real-time PCR(2) | Norovirus(type A) | F | 88 | 84 | D |
| | | R | | | |
| real-time PCR(2) | Norovirus(type B) | F | 88 | 85 | D |
| | | R | | | |
| real-time PCR(2) | Porcine astrovirus(types 1, 3, and 5) | F | 183 | 85 | D |
| | | R | | | |
| real-time PCR(2) | Swine influenza virus(H1N1 type) | F (SEQ ID NO 7) | 69 | 84 | D |
| | | R (SEQ ID NO 8) | | | |
| real-time PCR(2) | Swine influenza virus(H3N2 type) | F | 71 | 79 | D |
| | | R | | | |
| nested PCR(1) | Porcine enterovirus(type B) | F | 484 | | D |
| | | R | | | |
| | | nested F | 306 | | |
| | | nested R | | | |

TABLE 1-2

| | | | Size of | | |
|---|---|---|---|---|---|
| Reaction composition | | | PCR | Melting | |
| and condition used for | | Type of | product | temperature | Positive |
| PCR | Virus name | primer used | (bp) | (° C.) | control |
| standard PCR(2) | Sapovirus(type 3) | F | 330 | | D |
| | | R | | | |
| real-time PCR(2) | Sapovirus(type 6) | F | 67 | 80 | D |
| | | R | | | |
| standard PCR(2) | Sapovirus(type 7A) | F | 330 | | D |
| | | R | | | |
| standard PCR(2) | Sapovirus(type 7B) | F | 330 | | D |
| | | R | | | |
| standard PCR(2) | Sapovirus(type 7C) | F | 330 | | D |
| | | R | | | |
| real-time PCR(2) | Sapovirus(type 8) | F | 77 | 81 | D |
| | | R | | | |
| real-time PCR(2) | Sapovirus(type 9) | F | 70 | 83 | D |
| | | R | | | |
| | Sapovirus(type 10) | F | 112 | 83 | D |
| | | R | | | |
| standard PCR(4) | Vesicular exanthema of swine virus | F | 350 | | D |
| | | R | | | |
| real-time PCR(2) | Swine vesicular disease virus | F | 106 | 85 | D |
| | | R | | | |
| real-time PCR(2) | Porcine transmissible gastroenteritis virus(*1) | F | 113 | 78 | D |
| | | R | | | |
| real-time PCR(2) | Porcine Torque teno virus(type 1) | F | 90 | 62 | D |
| | | R | | | |
| real-time PCR(2) | Porcine Torque teno virus(type 2) | F | 64 | 82 | D |
| | | R | | | |
| standard PCR(5) | Porcine endogenous retrovirus(gag) | F | 337 | | |
| | | R | | | |
| standard PCR(5) | Porcine endogenous retrovirus(pol) | F | 810 | | |
| | | R | | | |
| real-time PCR(2) | Porcine endogenous retrovirus(pol) | F | 101 | 77 | |
| | | R | | | |
| standard PCR(5) | Porcine endogenous retrovirus(env A) | F | 359 | | |
| | | R | | | |
| standard PCR(5) | Porcine endogenous retrovirus(env B) | F | 263 | | |
| standard PCR(5) | Porcine endogenous retrovirus(env C) | R | 281 | | |
| | | F | | | |
| | | R | | | |
| real-time PCR(2) | Porcine reproductive and respiratory syndrome virus | F (SEQ ID NO 3) | 183 | 86 | A |
| | | R (SEQ ID NO 4) | | | |
| standard PCR(1) | Porcine epidemic diarrhea virus | F | 377 | | B |
| | | R | | | |
| real-time PCR(2) | Porcine rubulavirus | F | 95 | 84 | D |
| | | R | | | |
| standard PCR(6) | Porcine rotavirus(type A) | F | 309 | | D |
| | | R | | | |
| real-time PCR(2) | Porcine rotavirus(type B US variant) | F | 148 | 80 | D |
| | | R | | | |
| real-time PCR(2) | Porcine rotavirus(type B Vietnam variant) | F | 160 | 80 | D |
| | | R | | | |
| real-time PCR(2) | Porcine rotavirus(type C) | F | 75 | 78 | D |
| | | R | | | |
| nested PCR(1) | Mammalian reovirus | F | 416 | | D |
| | | R | | | |
| | | nested F | 344 | | |
| | | nested R | | | |
| real-time PCR(2) | Bornavirus | F | 120 | 87 | D |
| | | R | | | |
| real-time PCR(2) | Menangle virus | F | 66 | 82 | D |
| | | R | | | |
| | DNA virus | | | | |
| real-time PCR(2) | African swine fever virus | F | 120 | 82 | D |
| | | R | | | |
| real-time PCR(2) | Infectious bovine rhinotracheitis virus | F | 97 | 88 | D |
| | | R | | | |
| real-time PCR(2) | Aujeszky's disease virus | F | 61 | 85 | D |
| | | R | | | |
| real-time PCR(2) | Porcine adenovirus | F | 68 | 84 | D |
| real-time PCR(2) | Porcine cytomegalovirus | R | 64 | 86 | D |
| | | F (SEQ ID NO 11) | | | |
| | | R (SEQ ID NO 12) | | | |
| real-time PCR(2) | Porcine circovirus(type 1) | F | 64 | 79 | D |
| | | R | | | |
| real-time PCR(2) | Porcine circovirus(type 2) | F (SEQ ID NO 5) | 98 | 84 | A |
| | | R (SEQ ID NO 6) | | | |
| real-time PCR(2) | Porcine parvovirus | F | 75 | 81 | D |
| | | R | | | |
| real-time PCR(2) | Swine poxvirus | F | 78 | 77 | D |
| | | R | | | |
| real-time PCR(2) | Porcine lymphotropic herpesvirus(types 1 and 2) | F (SEQ ID NO 9) | 69 | 76 | D |
| | | R (SEQ ID NO 10) | | | |
| | protozoon | | | | |
| real-time PCR(2) | Toxoplasma gondii | F | 119 | 83 | D |
| | | R | | | |

(*1 )Primers for the porcine transmissible gastroenteritis virus are the same as for the respiratory coronavirus.

For viruses and protozoa, primers specific for pathogens shown in Table 1 above were used. Tm values of the primers fall within a range of 60° C. to 75° C. In the table above, the “Reaction composition and condition used for PCR” corresponds to Tables 2 and 3 below. In the table above, F and R primers used in the 2nd PCR in a nested PCR for improving specificity and a yield are described as nested F and R, respectively. In the table above, “positive control” is as follows:

A: a nucleic acid extracted from a target pathogen;
B: a nucleic acid extracted from Nisseiken PED live vaccine (manufactured by Nisseiken Co., Ltd.);
C: a nucleic acid extracted from SWINE ABNORMAL BIRTH 3 COMBO LIVE VACCINE (manufactured by Kyoto Biken Laboratories, Inc.); and
D: an artificial synthetic DNA.

For bacteria, all bacteria were targeted by using universal primers targeting a conserved region of a 16 rRNA gene (SEQ ID NOs: 1 and 2). The above-mentioned specific primers were used in this Example only if the following two conditions were met:

Condition 1: Wide coverage of a plurality of strains of a target pathogen rather than a single strain. About 50 sequences from the whole genome and genes targeted by the primers of the target pathogen were acquired from Genbank, aligned using CLC Main Workbench (manufactured by QIAGEN), and then confirmed that mismatches of the primers to all sequences were up to 3 bases. Note that, all registration data were acquired in the case of a pathogen of which only 50 or fewer sequences were registered in Genbank. Condition 2: High specificity for a target pathogen. Basic Local Alignment Search Tool (BLAST (registered trademark)) (manufactured by the National Institutes of Health) was used to confirm that the primers did not have homology to genes other than the target pathogen or a pig gene. Previously reported specific primers were preferentially employed in principle, and when the above-mentioned two conditions were not met, a consensus sequence in a highly conserved region in the above-mentioned alignment was selected and inputted into Universal Probe Library Assay Design Center (manufactured by Roche), which was a web-based software for preparing primers, to thereby design primers.

### (1.2. Positive Control for PCR)

Any of those described below was preferentially used in the following order as the positive control shown in the Table 1 above:

(1) a nucleic acid extracted from a target pathogen;
(2) a nucleic acid extracted from a commercially available live vaccine against a target pathogen; and
(3) an artificial synthetic DNA.

Viruses of which nucleic acids were used as the positive control were porcine reproductive and respiratory syndrome virus, porcine circovirus (type 2), and swine influenza virus (type H1N1), all of which are RNA viruses. For use as the positive control, RNA was extracted from pig plasma, which had been determined by a veterinarian at a general farm as being likely to be positively infected with the pathogens, using QIAamp Viral RNA kit (manufactured by QIAGEN) according to the protocol attached with the kit. However, a carrier RNA was not used upon RNA extraction.

cDNA was synthesized using SuperScript (registered trademark) II Reverse Transcriptase (manufactured by Thermo Fisher Scientific). A 12 μL reaction solution was prepared by including 1.5 μL of Random Primers (manufactured by Thermo Fisher Scientific), 4 μL of 2.5 mM dNTP Mix, 1 μL of RNA (250 ng/μL), and 5.5 μL of Nuclease Free Water (NFW). The reaction solution was left to stand at 65° C. for 5 minutes and transferred onto ice immediately. Subsequent procedures were performed according to the protocol attached to the kit. This cDNA was subjected to PCR-amplification by the previously reported method (Inoue R, Tsukahara T, Sunaba C, et al.: 2007, Simple and rapid detection of the porcine reproductive and respiratory syndrome virus from pig whole blood using filter paper. J Virol Methods 141: 102-106., Olvera A, Sibila M, Calsamiglia M, et al.: 2004, Comparison of porcine circovirus type 2 load in serum quantified by a real time PCR in postweaning multisystemic wasting syndrome and porcine dermatitis and nephropathy syndrome naturally affected pigs. J Virol Methods 117: 75-80.), sequenced with a sequence analysis, and then used as the positive control. Note that, swine influenza virus (type H1N1) was processed in the same manner as for other pathogens described above, except that a total of 10 μL of a reaction solution was prepared by mixing 5 μL of LightCycler (registered trademark) 480 Probes Master, 0.1 μL of Probe #104 (manufactured by Roche), 0.4 μL of each of 10 μM specific primer sets shown in the Table 1, 3.1 μL of NFW, and 1 μL of a template nucleic acid.

For bacteria, DNA extracted from Competent Quick Dh5a (Green Cap) (manufactured by TOYOBO CO., LTD.) using Quick Gene DNA tissue kit S (manufactured by Kurabo Industries Ltd.) was used. RNA extraction from a commercially available live vaccine and cDNA synthesis were performed in the same manner as described above.

For preparation of the artificially synthesized DNA, a sequence in which about 30 bp was added to each of upstream and downstream of a target region of primers in the consensus sequence mentioned in the above section (1.1. Primer) was synthesized as gBlocks (registered trademark) Gene Fragments (manufactured by INTEGRATED DNA TECHNOLOGIES). Protein regions coded by artificially synthesized DNAs corresponding to pathogens (apoi virus, hepatitis E virus (ORF1), hepatitis E virus (ORF2), bovine viral diarrhea virus (type 1), bovine viral diarrhea virus (type 2), bovine viral diarrhea virus (type 3), rabies virus (type 4), rabies virus (type 5), rabies virus (type 6), rabies virus (type 7), foot-and-mouth disease virus, respiratory coronavirus, sapovirus (type 3), sapovirus (type 6), sapovirus (type 7A), sapovirus (type 7B), sapovirus (type 7C), sapovirus (type 8), sapovirus (type 9), sapovirus (type 10), vesicular stomatitis virus (New Jersey variant), Western equine encephalitis virus, hemagglutinating encephalomyelitis virus, swine fever virus, Nipah virus, encephalomyocarditis virus, norovirus (type A), norovirus (type B), porcine astrovirus (type 1), porcine astrovirus (type 3), porcine astrovirus (type 5), swine influenza virus (type H1N1), swine influenza virus (type H3N2), porcine enterovirus (type B), vesicular exanthema of swine virus, swine vesicular disease virus, porcine transmissible gastroenteritis virus, porcine Torque teno virus (type 1), porcine Torque teno virus (type 2), rubulavirus, porcine rotavirus (type A), porcine rotavirus (B US variant), porcine rotavirus (B Vietnam variant), porcine rotavirus (type C), mammalian reovirus, bornavirus, Menangle virus, African swine fever virus, infectious bovine rhinotracheitis virus, Aujeszky's disease virus, porcine adenovirus, porcine cytomegalovirus, porcine circovirus (type 1), porcine parvovirus, swine poxvirus, porcine lymphotropic herpesvirus (types 1 and 2), Toxoplasma gondii) were particular regions in genes of the pathogens.

### (1.3. Work Flow for Establishing Detection System)

As an evaluation of a detection system, Conditions 1 to 3 below were confirmed to be met. Condition 1: when a PCR reaction was performed using a positive control gene suspended in NFW as a positive control and NFW as a negative control, a product of interest was amplified only in the positive control and a non-specific product was not amplified in the negative control. Condition 2: only target genes were also amplified in a positive control mixed solution (in NFW) in which equal amounts of positive control genes of about 10 types of pathogens were mixed. Condition 3: a PCR reaction was not interfered with by a pig-derived nucleic acid when pig pancreas-derived cDNA, which had been previously confirmed that the target gene was not amplified therefrom, was mixed with the positive control gene.

### (1.4. Detection of Virus by Standard PCR)

The pathogens were detected by a standard PCR using LifeECO (manufactured by NIPPON Genetics Co, Ltd). Two types of reaction compositions and six types of reaction conditions of the PCR used in this study are described in Tables 2 and 3 below, respectively. After amplification, the resultant products were electrophoresed on 1.5% agarose gel using a Tris-borate-EDTA buffer stock solution (manufactured by NACALAI TESQUE, INC.) to thereby confirm correspondence to a target size.

TABLE 2
Table. Reaction compositions of standard PCR and nested PCR

| | | | Standard PCR(1)(2)(5)(6) | |
|---|---|---|---|---|
| | Standard PCR(3)(6) | | and nested PCR(1)(2) | |
| GoTaq ® Greet Master Mix | 10 | μl | | |
| (manufactured by Promega) | | | | |
| 2 × KAPA HiFi Hot Star Ready Mix | | | 10 | μl |
| (manufactured by NIPPON Genetics Co, Ltd) | | | | |
| 10 μM primer set | Each 0.6 | μL | Each 0.6 | μL |
| Template nucleic acid | 1 | μl | 1.5 | μl |
| Nuclease Free Water | 7.8 | μl | 7.3 | μl |
| Total | 20 | μl | 20 | μl |

TABLE 3
Table. Conditions for PCR reaction

| | | | | | | | Final | Melting |
|---|---|---|---|---|---|---|---|---|
| | Initial | | Heat | | Elongation | Number | elongation | curve |
| Method | denaturation | | denaturation | Annealing | reaction | of cycles | reaction | analysis |
| standard PCR(1) | 95° C. 10 | min | 95° C. 20 sec | 55° C. 20 sec | 72° C. 25 sec | 35 | 72° C. 10 min | |
| standard PCR(2) | 95° C. 30 | sec | 95° C. 5 sec | 54° C. 20 sec | 75° C. 20 sec | 40 | 72° C. 10 min | |
| standard PCR(3) | 95° C. 10 | min | 95° C. 15 sec | 53° C. 10 sec | 72° C. 20 sec | 40 | 72° C. 10 min | |
| standard PCR(4) | 95° C. 5 | min | 95° C. 20 sec | 60° C. 15 sec | 72° C. 15 sec | 35 | 72° C. 10 min | |
| standard PCR(5) | 94° C. 10 | min | 94° C. 30 sec | 65° C. 45 sec | 72° C. 40 sec | 30 | 72° C. 10 min | |
| standard PCR(6) | 94° C. 5 | min | 94° C. 30 sec | 54° C. 30 sec | 72° C. 40 sec | 35 | 72° C. 10 min | |
| nested PCR(2) | | | | | | | | |
| nested PCR(1) | 95° C. 10 | min | 98° C. 30 sec | 60° C. 30 sec | 72° C. 40 sec | 30 | 72° C. 10 min | |
| real-time PCR(1) | 95° C. 30 | sec | 95° C. 5 sec | 58° C. 20 sec | 75° C. 20 sec | 40 | | 95° C. 5 sec, |
| | | | | | | | | 65° C. 60 sec |
| real-time PCR(2) | 95° C. 30 | sec | 95° C. 5 sec | | 60° C. 30 sec | 40 | | 95° C. 5 sec, |
| | | | | | | | | 65° C. 60 sec |
| real-time PCR(3) | 95° C. 30 | sec | 95° C. 5 sec | 55° C. 20 sec | 75° C. 20 sec | 40 | | 95° C. 5 sec, |
| | | | | | | | | 65° C. 60 sec |

### (1.5. Detection of Virus by Nested PCR)

The LifeECO was used in the same manner as in the above section (1.4. Detection of virus by standard PCR). The same reaction composition shown in the Table 2 above was used in both the 1st PCR and the 2nd PCR. As a template nucleic acid in the 2nd PCR, a PCR product of the 1st PCR was directly used without dilution or purification. The same reaction conditions shown in the Table 3 above were used in both 1st PCR and 2nd PCR. The amplified product was confirmed by electrophoresis in the same manner as in the above section (1.4. Detection of virus by standard PCR).

### (1.6. Detection of Virus and Protozoon by Real-Time PCR)

Light Cycler (registered trademark) 480 (manufactured by Roche) was used. A total of 10 μL of a reaction solution was prepared by mixing 5 μL of SYBR (registered trademark) Premix Ex Taq II (Tli RNase Plus) (manufactured by Takara Bio Inc.), 0.2 μL of each of 10 μM primer sets, 3.6 μL of NFW, and 1 μL of a template nucleic acid. The reaction was performed in duplicate. The reaction conditions for each pathogen are shown in the Table 3 above. After melting curve analysis, the PCR product was electrophoresed on 2% agarose gel (Tris-borate-EDTA) to thereby confirm correspondence to a target size.

### (1.7. Detection of Bacteria)

For detection of bacteria, 341F: 5′-CCTACGGGNGGCWGCAG-3′ (Tm value: 68° C.; SEQ ID NO: 1) and 805R: 5′-GACTACHVGGGTATCTAATCC-3′ (Tm value: 56.5° C.; SEQ ID NO: 2) (Klindworth A et al.,2013) were used as primers targeting a highly conserved region of a 16s rRNA gene (v3−v4 region). The PCR was performed in a total of 25 μL of a reaction solution containing 12.5 μL of KAPA HiFi Hot Start Ready Mix (manufactured by NIPPON Genetics Co, Ltd), 5 μL of each of 0.2 μM primer sets, and 2.5 μL of DNA. After initial denaturation at 95° C. for 3 minutes, heat denaturation at 95° C. for 30 seconds, annealing at 55° C. for 30 seconds, and an elongation reaction at 72° C. for 30 seconds were performed for 35 cycles. Thereafter, a final elongation reaction at 72° C. for 5 minutes was performed. The amplified product was confirmed by electrophoresis in the same manner as in the above section

### (1.4. Detection of Virus by Standard PCR).

### (1.8. Measurement of Detection Limit Copy Number)

### ((Preparation of Plasmid for Positive Control))

For a detection system using, as a positive control, a nucleic acid extracted from the target pathogen or a commercially available live vaccine against the target pathogen, a plasmid including DNA serving as the positive control was obtained as described below in order to understand the accurate copy number of the DNA. The PCR product obtained as mentioned above was purified using Wizard (registered trademark) SV Gel and PCR Clean-Up System (manufactured by Promega). An equal amount of Membrane Binding Solution was mixed with the PCR product and a total amount thereof was added to Econo Spin (registered trademark) for DNA (manufactured by Epoch Biolabs). After leaving to stand at room temperature for 1 minute, the resultant was centrifuged at 16,000×g at room temperature for 1 minute. The resultant filtrate was removed, added with 400 μL of Membrane Wash Solution, and centrifuged under the same conditions as the previous centrifugation. The resultant filtrate was removed again, added with 400 μL of Membrane Wash Solution, and centrifuged at 16,000×g at room temperature for 5 minutes. Another tube was set on a column, added with 30 μL of NFW, and left to stand at room temperature for 1 minute. The resultant was centrifuged at 16,000×g at room temperature for 1 minute. Thereafter, TOPO TA (registered trademark) Cloning (registered trademark) kit (manufactured by Thermo Fisher Scientific) was used to incorporate the resultant into a cloning vector attached with the kit. Two microliters of the vector was transformed into Competent Quick Dh5a (Green Cap) (manufactured by TOYOBO CO., LTD.) using a heat shock method and seeded into an LB medium. Grown colonies were screened via colony PCR for insertion of the insert. GoTaq (registered trademark) Green M aster Mix (manufactured by Promega) was used for the colony PCR and a total of 25 μL of a reaction solution was prepared including 12.5 μL of Master Mix, 0.5 μL of each of M13 Forward Primer and M13 Reverse Primer (10 μM) attached with TOPO TA (registered trademark) Cloning (registered trademark) kit (manufactured by Thermo Fisher Scientific), and 11.5 μL of NFW. The colony was gently touched with a 10 μL pipette tip and added to the reaction solution. After initial denaturation at 94° C. for 2 minutes, heat denaturation at 98° C. for 10 seconds, annealing at 55° C. for 30 seconds, and an elongation reaction at 72° C. for 30 seconds were performed for 30 cycles. Electrophoresis was performed in the same manner as in the above section (1.4. Detection of virus by standard PCR). The colony from which the presence of the band having confirmation of a target size was seeded into 6 mL of LB broth containing 50 μg/mL ampicillin and cultured at 37° C. overnight. Mini Plus (registered trademark) Plasmid DNA Extraction System (manufactured by VIOGENE) was used to extract a plasmid according to the protocol attached with the kit. The plasmid was measured for concentration using NanoDrop (registered trademark) 1000 Spectrophotometer (manufactured by Thermo Fisher Scientific) to calculate the copy number.

### ((Measurement of Detection Limit Copy Number))

Only viruses screened for the presence or the possibility of the presence within the country were measured for a detection limit copy number. Six samples were prepared in 10-fold dilution series so as to have a maximum concentration of about 1 to 9×10⁵ copies/μL and a minimum concentration of about 1 to 9 copies/μL and subjected to the PCR under the conditions for respective pathogens described in the above section (1.4. Detection of virus by standard PCR) to the above section (1.6. Detection of virus and protozoon by real-time PCR). Only the real-time PCR was performed in duplicate, and if a product of interest was confirmed to be amplified in one of the duplicate reactions at a dilution step which was a limit in judgment of the detection limit, it was considered positive. For the real-time PCR, PCR efficiency was calculated using the expression below. Note that, the term slope in the expression refers to a slope on a graph where the vertical axis represents the number of cycles and the horizontal axis represents the Log copy number.

$$PCR efficiency = {10}^{- \frac{1}{slope}} - 1 \qquad [ Formula 1 ]$$

### <2. Evaluation of PCR Testing Systems Using Clinical Specimens>

### (2.1. Clinical Specimen)

As clinical specimens, specimens which were considered to have high cleanliness (Clean sample) and a specimen which was considered to have low cleanliness, that is, to be highly likely to be infected with a pathogen (Infected sample) were prepared. As the Clean sample, the livers, the kidneys, the pancreases, and the blood from two 21-week-old piglets which have been raised as DPF candidate pigs using the U-iR method were used (Clean samples 1 and 2). As the Infected sample, the liver, the kidney, the lung, and the lymph node from a pig which had been dissected and inspected for suspicion of an infectious disease by a veterinarian at a general farm was used.

(2.2. RNA Extraction from Organs)

Fifty milligrams of the organ was chopped using ophthalmologic scissors, added with 1 mL of TRIzol (registered trademark) LS Reagent (manufactured by life technologies), and then homogenized with Tissue Ruptor II (manufactured by QIAGEN). Two-hundred microliters of chloroform (manufactured by Wako) was added thereto, vortexed for 15 seconds, and then incubated at room temperature for 2 minutes. The resultant was centrifuged at 12,000×g at room temperature for 15 minutes and then 350 μL of a transparent upper layer (aqueous layer) was collected into another tube. RNeasy (registered trademark) Mini kit (manufactured by QIAGEN) was used to purified RNA from the collected upper layer.

The RNA was added with 70% (v/v) ethanol in an amount equal to the collected upper layer, mixed by inverting, and then a total amount of the mixed solution was applied into a column attached with the kit. The resultant was centrifuged at 8,000×g at room temperature for 15 minutes and the filtrate was discarded. The column was added with 350 μL of Buffer RW1 and centrifuged under the same conditions as the previous centrifugation, and then the filtrate was discarded. The resultant was treated with DNase using RNase-Free DNase Set (manufactured by QIAGEN). A solution in which DNaseI and Buffer RDD, which were attached with the kit, were suspended at 1:7 was added in an amount of 75 μL and left to stand at room temperature for 8 minutes. The resultant was added with 350 μL of RW1 and centrifuged under the same conditions as the previous centrifugation, and then the filtrate was discarded. The resultant was added with 500 μL of Buffer RPE and centrifuged under the same conditions as the previous centrifugation, and then the filtrate was discarded. The resultant was added with 500 μL of Buffer RPE again and centrifuged at 8,000×g at room temperature for 2 minutes, and the filtrate was discarded. An empty column was centrifuged at 20,000×g at room temperature for 1 minute. Another tube was set on a column, added with 50 μL of RNase Free Water, left to stand for 1 minute, and then centrifuged at 20,000×g at room temperature for 1 minute.

(2.3. RNA Extraction from Blood)

Seven hundred and fifty microliters of TRIzol (registered trademark) LS Reagent (manufactured by life technologies) was added to 250 μL of heparinemia, vortexed, and left to stand at room temperature for 5 minutes. Subsequent procedures were performed in the same manner as in the above section (2.2. RNA extraction from organs) except for the following: For RNA extraction from blood, chloroform was added; 560 μL of the upper layer (aqueous layer) was collected after centrifugation; the DNase treatment was not performed; and washing with 500 μL of 80% (v/v) ethanol was performed instead of the second washing with the Buffer RPE. Furthermore, the empty column was centrifuged at 20,000×g at room temperature for 5 minutes prior to elution, and elution was performed with 30 μL of RNase Free Water.

(2.4. DNA Extraction from Organs and Blood)

DNA was extracted from organs using Quick Gene DNA tissue kit S (manufactured by Kurabo Industries Ltd.) in the same manner as in the above section (1.2. Positive control for PCR). DNA was extracted from heparinemia using QIAamp (registered trademark) DNA Blood Mini Kit (manufactured by QIAGEN) according to the protocol attached with the kit.

### (2.5. PCR)

For detection of RNA viruses, cDNA was prepared in the same manner as in the above section (1.2. Positive control for PCR). Furthermore, cDNA was also prepared using Oligo dT₂₀ (manufactured by Thermo Fisher Scientific) as a primer for reverse transcription. DNA viruses and RNA viruses were detected using the DNA and the cDNA reverse-transcribed with the above-mentioned two types of primers, respectively, in the same manner as in the above section (1.4. Detection of virus by standard PCR) to the above section (1.7. Detection of bacteria). Note that, for the Infected sample, a mixture of equal amounts of cDNA and DNA from each organ was used as a template.

### <3. Result>

### (3.1. Detection of Pathogen by PCR)

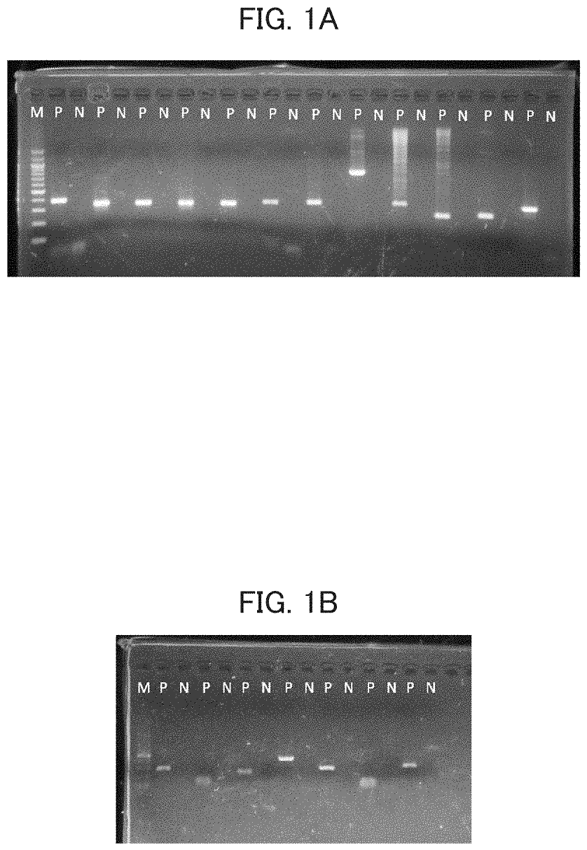

Sixty-three types of viruses and one type of protozoon shown in the Tables 1-1 and 1-2 were confirmed to meet Conditions 1 to 3 described in the above section (1.3. Work flow for establishing detection system). FIG. 1 shows electrophoresis results of PCR products of 15 types of viruses detected by the standard PCR and the nested PCR.

FIG. 1A shows electrophoresis results of the PCR products as detection results by the standard PCR. In the figure, P refers to a positive control which was a mixture of a pig pancreas-derived cDNA, which had been previously confirmed that the target gene was not amplified therefrom, with a positive control gene; and N refers to a negative control which was cDNA which was the same as those mentioned above. A 100 bp ladder marker (M) is shown at the left end and subsequently getah virus, sapoviruses (type 3), (type 7A), (type 7B), and (type 7C), vesicular exanthema of swine virus, porcine endogenous retrovirus (PERV) (gag), (pol), (envA), (envB), and (envC), and porcine epidemic diarrhea virus are shown by two wells for each. Target sizes were 380 bp, 330 bp, 330 bp, 330 bp, 330 bp, 350 bp, 337 bp, 810 bp, 359 bp, 263 bp, 281 bp, and 377 bp, respectively. FIG. 1B shows electrophoresis results of 2nd PCR products as detection results by the nested PCR. In the figure, P and N were the same as in FIG. 1A. The result of a 100 bp ladder marker (M) is shown at the left end and subsequently, the results of hepatitis E virus (ORF1), hepatitis E virus (ORF2), rabies virus (type 7), hemagglutinating encephalomyelitis virus, porcine enterovirus, porcine rotavirus (type A), and mammalian reovirus are shown by two wells for each. Target sizes were 287 bp, 145 bp, 259 bp, 472 bp, 306 bp, 121 bp, and 344 bp, respectively.

Furthermore, melting curves were obtained for 48 types of viruses detected by the real-time PCR (African swine fever virus, apoi virus, bovine viral diarrhea virus type 1, bovine viral diarrhea virus type 2, bovine viral diarrhea virus type 3, infectious bovine rhinotracheitis virus, Aujeszky's disease virus, rabies virus 4, rabies virus 5, rabies virus 6, foot-and-mouth disease virus, porcine respiratory coronavirus, sapovirus type 6, sapovirus type 8, sapovirus type 9, sapovirus type 10, vesicular stomatitis virus (New Jersey variant), Western equine encephalitis virus, swine fever virus, Nipah virus, encephalomyocarditis virus, norovirus type A, norovirus type B, porcine astrovirus type 1, porcine astrovirus type 3, porcine astrovirus type 5, porcine adenovirus, swine influenza virus type H1N1, swine influenza virus type H3N2, porcine cytomegalovirus, porcine circovirus type 1, porcine circovirus type 2, swine vesicular disease virus, porcine transmissible gastroenteritis virus, porcine Torque teno virus type 1, porcine Torque teno virus type 2, porcine endogenous retrovirus (pol gene), porcine parvovirus, porcine reproductive and respiratory syndrome virus, swine poxvirus, porcine lymphotropic herpesvirus type 1, porcine lymphotropic herpesvirus type 2, porcine rubulavirus, porcine rotavirus B US variant, porcine rotavirus B Vietnam variant, porcine rotavirus C, bornavirus, Toxoplasma gondii, Menangle virus, etc.). The figure is omitted. A pig pancreas-derived cDNA which had been previously confirmed that the target gene was not amplified therefrom was used as a negative control and a mixture of the cDNA with a positive control gene was used as a positive control. Note that, for the porcine endogenous retrovirus (pol gene), NFW was used as a negative control.

### (3.2. Detection Limit Copy Number)

The detection limit copy numbers of the PCRs were shown in Table 4 to one significant digit. Seven types of viruses were measured for the detection limit by the standard PCR, and the porcine epidemic diarrhea virus, the porcine rotavirus (type A), the getah virus, the sapovirus (type 7A, type 7B), the Japanese encephalitis virus, and the sapovirus (type 3, type 7C) had the detection limit of 200 copies/μL, 300 copies/μL, 2,000 copies/μL, 2,000 copies/μL, 2,000 copies/μL, and 20,000 copies/μL, respectively. Five types of viruses were measured by the nested PCR, of which two types, the hepatitis E virus (ORF2) and the mammalian reovirus had the detection limit of 50 copies/μL or less and the remaining three types had the detection limit of 1,000 to 2,000 copies/μL. Meanwhile, for the real-time PCR, the infectious bovine rhinotracheitis virus had the detection limit of 4,000 copies/μL, which was higher than those of other pathogens, that is, 20 to 700 copies/μL, by one order of magnitude. The Torque teno virus (type 1), the swine fever virus, the norovirus (type A), and the norovirus (type B) had the PCR efficiency of 58%, 55%, 54%, and 56%, respectively, which was lower than those of other pathogens, that is 63% to 96%.

| | Detection limit | | |
|---|---|---|---|
| | copy numbers | PCR | |
| | (copies/μl) | efficiency | Method |
| Virus | | — | |
| Getah virus | 2,000 | — | standard PCR |
| Sapovirus(type 3) | 20,000 | — | standard PCR |
| Sapovirus(type 7A) | 2,000 | — | standard PCR |
| Sapovirus(type 7B) | 2,000 | — | standard PCR |
| Sapovirus(type 7C) | 20,000 | — | standard PCR |
| Japanese encephalitis virus | 2,000 | — | standard PCR |
| Porcine epidemic diarrhea virus | 200 | — | standard PCR |
| Porcine rotavirus (type A) | 300 | — | standard PCR |
| Hepatitis E virus (ORF1) | 2,000 | — | nested PCR |
| (ORF2) | 50 | — | nested PCR |
| Hemagglutinating encephalomyelitis virus | 1,000 | — | nested PCR |
| Porcine enterovirus | 2,000 | — | nested PCR |
| Mammalian reovirus | 10 | — | nested PCR |
| African swine fever virus | *20 | 82% | real-time PCR |
| Bovine viral diarrhea virus(type 1) | 200 | 73% | real-time PCR |
| Bovine viral diarrhea virus (type 2) | 400 | 79% | real-time PCR |
| Bovine viral diarrhea virus (type 3) | 60 | 66% | real-time PCR |
| Bovine viral diarrhea virus | 4,000 | 76% | real-time PCR |
| Aujeszky's disease virus | 400 | 83% | real-time PCR |
| Foot-and-mouth disease virus | 500 | 74% | real-time PCR |
| Respiratory coronavirus | 500* | 66% | real-time PCR |
| Sapovirus(type 6) | 700 | 71% | real-time PCR |
| Sapovirus(type 8) | 30 | 96% | real-time PCR |
| Sapovirus(type 9) | 600 | 85% | real-time PCR |
| Sapovirus(type 10) | 400 | 82% | real-time PCR |
| Swine fever virus | 45* | 55% | real-time PCR |
| Encephalomyocarditis virus | 700 | 82% | real-time PCR |
| Norovirus(type A) | 400* | 55% | real-time PCR |
| Norovirus(Type B) | 400* | 56% | real-time PCR |
| Swine influenza virus(H1N1 type) | 70* | 82% | real-time PCR |
| Swine influenza virus(H3N2 type) | 40 | 63% | real-time PCR |
| Porcine cytomegalovirus | 700 | 87% | real-time PCR |
| Porcine circovirus(type 1) | 10 | 96% | real-time PCR |
| Porcine circovirus(type 2) | 100 | 82% | real-time PCR |
| Porcine transmissible gastroenteritis virus | 500* | 66% | real-time PCR |
| Porcine Torque teno virus(type 1) | 600* | 58% | real-time PCR |
| Porcine Torque teno virus(type 2) | 60 | 94% | real-time PCR |
| Porcine parvovirus | 400 | 85% | real-time PCR |
| Porcine reproductive and respiratory syndrome virus | 10 | 92% | real-time PCR |
| Swine poxvirus | 30* | 68% | real-time PCR |
| Porcine lymphotropic herpesvirus(type 1) | 200 | 75% | real-time PCR |
| Porcine lymphotropic herpesvirus(type 2) | 200 | 75% | real-time PCR |
| Porcine rotavirus(type B US variant) | 400 | 74% | real-time PCR |
| Porcine rotavirus(type B Vietnam variant) | 40* | 77% | real-time PCR |
| Porcine rotavirus (type C) | 300 | 78% | real-time PCR |
| Protozoon | | | |
| Toxoplasma | 500 | 73% | real-time PCR |

*The real-time PCR was performed in duplicate and a product of interest was confirmed to be amplified in only one of the duplicate reactions at a limit dilution step.

For detection sensitivity of the pathogens, the nested PCR, of which the number of cycles was higher than that of the standard PCR by about 25, had higher detection sensitivity. Meanwhile, for most pathogens, the real-time PCR could be confirmed to have the detection sensitivity higher than that of the above-mentioned two PCR methods by about one order of magnitude. Therefore, it can be said that all detection is preferably performed by the real-time PCR.

### (3.3. Evaluation of PCR Using Clinical Specimens>

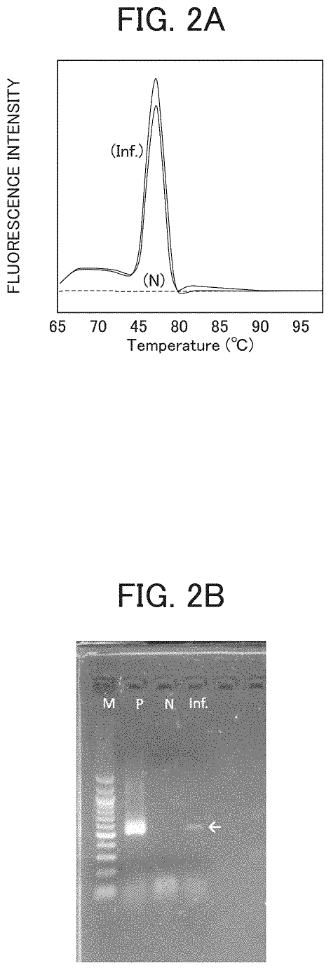

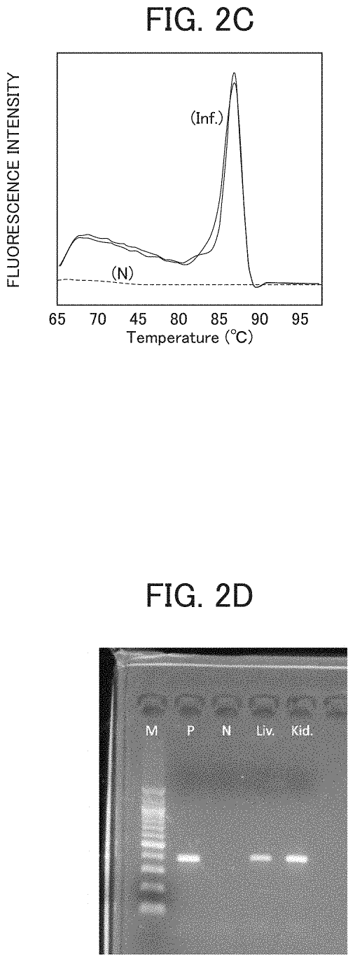

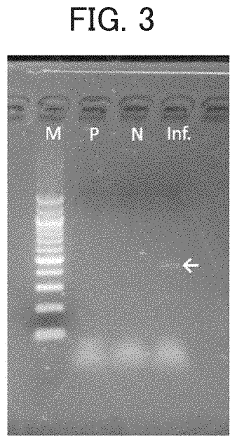

FIG. 2 shows detection results of pathogens for evaluating the PCR testing system using clinical specimens. FIG. 2A shows melting curves as the detection results of porcine lymphotropic herpesvirus for the Infected sample (Inf.) in duplicate and the negative control (N). FIG. 2B shows the electropherogram as the detection results of bacteria and shows, from the left, a 100 bp ladder marker (M), a positive control gene (P), NFW serving as a negative control (N), and the Infected sample (Inf.). The target size was 464 bp. FIG. 2C shows melting curves as the detection results of porcine cytomegalovirus for the Infected sample (Inf.) in duplicate and the negative control (N). FIG. 2D shows the electropherogram as the detection result of getah virus and shows, from the left, a 100 bp ladder marker (M), a positive control gene (P), NFW serving as a negative control (N), and the liver (Liv.) and the kidney (Kid.) of the Clean sample 1. The target size was 330 bp. The band was less visible in the case of 35 cycles as in Table 4, and, therefore, the results shown in this figure are the results with the increased number of cycles of 45.

The porcine endogenous retrovirus was detected in all organs from the Infected sample and the two Clean samples. Furthermore, as shown in FIG. 2, the porcine lymphotropic herpesvirus, bacteria, and the porcine cytomegalovirus were also detected in the Infected sample. The getah virus was detected in the Clean sample 1 only when Oligo dT₂₀ primer was used as a reverse transcriptase (FIG. 2). The PCR product was subjected to direct sequencing and confirmed to have homology to the getah virus. Furthermore, non-specific bands were visible in detection of the hepatitis E virus (ORF2), the rabies virus (type 7), and the vesicular exanthema of swine virus, so the PCR products were subjected to direct sequencing and confirmed to be RCR artifacts rather than the pathogens.

For the porcine cytomegalovirus, detection was also performed using primers reported in Hamel, A. L., L. Lin, C. Sachvie, E. Grudeski, and G. P. Nayer. 1999. PCR assay for detecting porcine cytomegalovirus. J. Clin. Microbiol. 37: 3767-3768, for confirmation. FIG. 3 shows the electropherogram of the PCR products of the cytomegalovirus using the reported primers and shows, from the left, a 100 bp ladder marker (M), a positive control gene (P), NFW serving as a negative control (N), and the Infected sample (Inf.). The target size is 413 bp and a band corresponding to the target size was confirmed.

### (3.4. Discussion)

For specimens using the clinical specimens, the test method according to the present invention was used to detect in the Infected sample the porcine lymphotropic herpesvirus, bacteria, and the porcine cytomegalovirus, in addition to the porcine endogenous retrovirus. The porcine lymphotropic herpesvirus and the porcine cytomegalovirus are widely spread in healthy pig groups and usually not apparent. Therefore, the pathogen which caused clinical symptoms such as diarrhea and bloating in the pig used as the Infected sample in the test can be identified as bacteria detected according to the present invention rather than the above-mentioned viruses. Both viruses are non-apparent viruses, and, therefore, are not particularly perceived as a problem in the general hog raising industry. However, it has been suggested that the porcine lymphotropic herpesvirus is involved in lymphoproliferative diseases after allotransplantation in pigs. Furthermore, pigs which do not have immunity against the porcine cytomegalovirus are difficult to breed. Although unlikely, the risk of mother-to-child transmission has been suggested for porcine lymphotropic herpesvirus. The mother-to-child transmission often occurs for porcine cytomegalovirus, and it was reported that the virus was isolated from 65% of piglets born from mothers experimentally infected during pregnancy. As mentioned above, the test method according to the present invention can detect a plurality of types of viral infections from clinical specimens as well as bacterial infections.

The detection system by PCR established in the above section <1. Establishment of system for detecting pathogen by PCR> can detect infection by various pathogens in the Infected sample and can confirm that pathogen infection is significantly low in the Clean sample, and, therefore, a xenotransplantation material with less pathogen infection can be provided. In particular, according to the test method, all 25 bacterial pathogens in principle included in the guidelines of the Ministry of Health, Labor and Welfare are covered by using a common primer.

What is claimed is:

1. A method for testing a xenotransplantation material for pathogen infection, the method comprising performing genetic tests for infection by pathogens comprising two or more types of bacteria and a virus or a protozoon, wherein the genetic test for the two or more types of bacteria comprises using at least one primer that targets a common base sequence among at least two types of bacteria.

2. The method according to claim 1, wherein the common base sequence is a sequence comprised in a conserved region of bacterial 16S rRNA.

3. The method according to claim 2, wherein the conserved region is a conserved region at least common to 16S rRNAs of Leptospira species, Mycoplasma species, Campylobacter species, Yersinia species, Escherichia species, and Salmonella species.

4. The method according to claim 2, wherein the conserved region is a region comprising V3 and V4 regions.

5. The method according to claim 1, wherein one primer pair is used in the genetic test for the two or more types of bacteria.

6. The method according to claim 1, wherein the genetic test for infection by the two or more types of bacteria and the genetic test for infection by the virus or the protozoon are performed by a PCR method with the same thermal cycle conditions using primers among which differences in melting temperature are 15° C. or less in any combination.

7. The method according to claim 1, wherein the pathogen infection causes a zoonotic disease.

8. The method according to claim 1, wherein performing the genetic test for infection by the virus is a testing for infection by five or more types of viruses by a PCR method using at least one primer pair that selectively binds to respective viral nucleic acids, or
performing the genetic test for infection by the protozoon is a testing for infection by two or more types of protozoa by a PCR method using at least one primer pair that selectively binds to respective protozoan nucleic acids.

9. The method according to claim 1, wherein the xenotransplantation material is a pig-derived xenotransplantation material.

10. The method according to claim 1, wherein the virus comprises at least one of porcine reproductive and respiratory syndrome virus, porcine circovirus type 2, swine influenza virus type H1N1, porcine lymphotropic herpesvirus, porcine cytomegalovirus, and hepatitis E virus, or the protozoon comprises Toxoplasma species.

11. A test kit, comprising:
at least one primer that targets a common base sequence among at least two types of bacteria; and
at least one primer that selectively binds to a nucleic acid of at least one type of a virus or a protozoon.

12. A method for producing a xenotransplantation material product which has been evaluated for pathogen infection, the method comprising:
performing the method according to claim 1.


SEQUENCE LISTING (16 sequences)

SEQ ID NO: 1 - DNA, 17 bp, Artificial Sequence
  341F primer
  misc_feature (9)..(9): n is a, c, g, or t
cctacgggnggcwgcag

SEQ ID NO: 2 - DNA, 21 bp, Artificial Sequence
  805R primer
gactachvgggtatctaatcc

SEQ ID NO: 3 - DNA, 20 bp, Artificial Sequence
  1primerF
cactgtggagtttagtttgc

SEQ ID NO: 4 - DNA, 23 bp, Artificial Sequence
  1primerR
cacacggtcgccctaattgaata

SEQ ID NO: 5 - DNA, 19 bp, Artificial Sequence
  2primerF
ccaggagggcgttgtgact

SEQ ID NO: 6 - DNA, 20 bp, Artificial Sequence
  2primerR
cgctaccgttggagaaggaa

SEQ ID NO: 7 - DNA, 20 bp, Artificial Sequence
  3primerF
aggatttgtgttcacgctca

SEQ ID NO: 8 - DNA, 20 bp, Artificial Sequence
  3primerR
gcattttggacaaagcgtct

SEQ ID NO: 9 - DNA, 23 bp, Artificial Sequence
  4primerF
aggacctgcataagtatcctcaa

SEQ ID NO: 10 - DNA, 21 bp, Artificial Sequence
  4primerR
cagggccagaactaatcaaaa

SEQ ID NO: 11 - DNA, 20 bp, Artificial Sequence
  5primerF
aatgcgttttacggcttcac

SEQ ID NO: 12 - DNA, 18 bp, Artificial Sequence
  5primerR
acgccgctagagggagac

SEQ ID NO: 13 - DNA, 24 bp, Artificial Sequence
  6primerF
ctggcatyactactgcyattgagc

SEQ ID NO: 14 - DNA, 23 bp, Artificial Sequence
  6primerR
ccatcrarrcagtaagtgcggtc

SEQ ID NO: 15 - DNA, 20 bp, Artificial Sequence
  7primerF
ctagggcacccttactgcaa

SEQ ID NO: 16 - DNA, 20 bp, Artificial Sequence
  7primerR
ctgctggatctcttcccttg